(19) United States
(12) Patent Application Publication
(10) Pub. No.: US 2025/0066795 A1
(43) Pub. Date: Feb. 27, 2025

(54) ACVR1R206H ALLELE-SPECIFIC THERAPY AND USES THEREOF FOR TREATMENT OF FIBRODYSPLASIA OSSIFICANS PROGRESSIVA

(71) Applicant: Oligomicstx Inc., Calgary (CA)

(72) Inventors: Toshifumi YOKOTA, Calgary (CA); Rika YOKOTA-MARUYAMA, Calgary (CA)

(21) Appl. No.: 18/713,681

(22) PCT Filed: Dec. 1, 2022
(86) PCT No.: PCT/CA2022/051756

§ 371 Date: May 27, 2024

Related U.S. Application Data
(60) Provisional application No. 63/285,547, filed on Dec. 3, 2021.

(51) Int. Cl. C12N 15/113; A61P 19/00

(52) U.S. Cl. CPC C12N 15/1138; A61P 19/00; C12N 2310/11; C12N 2310/313; C12N 2310/321; C12N 2310/3231; C12N 2310/341

(57) ABSTRACT

Described herein is the preferential knockdown of the mutant transcript ACVR1^{R206H} and inhibition of osteogenic differentiation using allele-selective gapmers for the treatment of Fibrodysplasia Ossificans Progressiva.

This application claims priority to U.S. Provisional Application Ser. No. 63/285,547 filed Dec. 3, 2021 which is hereby incorporated by reference in its entirety.

### FIELD OF THE INVENTION

The present invention relates to ACVR1^{R206H} allele-specific therapy and uses thereof for treatment of Fibrodysplasia ossificans progressiva (FOP) caused by ACVR1^{R206H}. In particular, the invention relates to therapeutics comprising gapmers for the preferential knockdown of the mutant transcript encoding ACVR1^{R206H} and inhibition of osteogenic differentiation using these therapeutics for the treatment of FOP.

### BACKGROUND

Fibrodysplasia ossificans progressiva (FOP) is a rare autosomal dominant disorder characterized by episodic heterotopic ossification in skeletal muscle, tendon, and the associated soft connective tissues[1]. Heterotopic ossification can occur in swelling of soft tissue (flare-up), which is often triggered by injuries, surgery, intramuscular injections, or viral infection [1].

FOP is caused by mutations in Activin A receptor, type I (ACVR1)/Activin receptor-like kinase-2 (ALK2), a bone morphogenetic protein (BMP) type I receptor. The majority of FOP mutations result in the amino acid substitution R206H within the glycine-serine domain of ACVR1 [2,3]. Ectopic activation of ACVR1^{R206H} by activin A induces heterotopic ossification [4, 5]. The R206H substitution renders ACVR1 responsive to activin A, which does not activate wild-type ACVR1 [4, 5]. Recent in vivo studies demonstrated that fibro/adipogenic progenitors are primary sources of heterotopic ossification in FOP mouse models [6, 7]. More than 95% of FOP is caused by a recurrent mutation of 617G>A.

Effective therapies for FOP are currently unavailable. Antibodies and small molecules targeting AVCR1, and other effectors including activin A and BMP pathway members are potential therapeutics. AVCR1 therapeutics that target both wild-type AVCR1 and FOP-mutated AVCR1 as well as those targeting other effectors by inhibiting BMP signaling may have undesirable impact on normal cellular and tissue function. A small molecule developed by Blueprint Medicines, Inc. (BLU-782; also known as IPN60130) selectively targets the FOP-mutated AVCR1 with minimal interference to the wild-type AVCR1.

There are however no therapeutics that selectively inhibit the mutant allele mRNA encoding the FOP-mutated AVCR1. Accordingly, there remains a need for allele-specific therapeutics that target mutant allele mRNA.

### SUMMARY OF THE INVENTION

An object of the present invention is to provide ACVR1^{R206H} allele-specific therapy and uses thereof for treatment of Fibrodysplasia ossificans progressiva (FOP) and other diseases caused by ACVR1^{R206H}.

In accordance with one aspect of the invention, there is provided a modified antisense oligonucleotide consisting of 12 to 30 linked nucleotides substantially complementary to a portion of exon 6 of an ACVR1 mutant allele encoding ACVR1^{R206H} mutant protein and comprising a wing-gap-wing motif with a 5′ wing region positioned at the 5′ end of a deoxynucleoside gap region, and a 3′ wing region positioned at the 3′ end of the deoxynucleoside gap region, wherein the 5′ wing region comprises at least three 2′ sugar modified nucleotides, the 3′ wing region comprises at least three 2′ sugar modified nucleotides and wherein the deoxynucleoside gap region comprises a sequence complementary to a portion of exon 6 of ACVR1 having the 617G>A mutation of the ACVR1 mutant allele.

In some embodiments of the invention, the gap region comprises the sequence 5′-CTGGTG-3′.

In some embodiments of the invention, the oligonucleotide comprises the sequence 5′-AATCTGGTG-3′.

In accordance with another aspect of the invention, there is provided a modified antisense oligonucleotide consisting of 12 to 30 linked nucleotides substantially complementary to a portion of exon 6 of an ACVR1 mutant allele encoding ACVR1^{R206H} mutant protein consisting of 12 to 30 linked nucleosides, wherein the modified oligonucleotide is capable of selectively reducing the expression of the mutant allele and comprises a wing-gap-wing motif with a 5′ wing region positioned at the 5′ end of a deoxynucleoside gap region, and a 3′ wing region positioned at the 3′ end of the deoxynucleoside gap, wherein the 5′ wing region comprises at least three locked nucleotides, the 3′ wing region comprises at least three locked nucleotides and wherein the deoxynucleoside gap region comprises a sequence complementary to a portion of exon 6 of ACVR1 having the 617G>A mutation of the ACVR1 mutant allele.

In accordance with another aspect of the invention, there is provided an antisense gapmer oligonucleotide that is at least 15 or 16 nucleotides in length, consisting of linked nucleosides complementary to an equal length portion of exon 6 of ACVR1, the antisense gapmer oligonucleotide consisting of three regions:
a 5′ region (5′R) consisting of at least three 2′ sugar modified nucleotides,
a gap region (GR) consisting of linked nucleotides,
a 3′ region (3′R) consisting of at least three 2′ sugar modified nucleotides, wherein the 5′ region (5′R), gap region (GR), or the 3′ region (3′R), comprises a sequence complementary to a portion of exon 6 of ACVR1 having the 617G>A mutation of the ACVR1 mutant allele ACVR1 encoding the ACVR1^{R206H}.

In accordance with another aspect of the invention, there is provided an antisense gapmer oligonucleotide having 3 nucleotide long 5′ and 3′ wings comprising locked nucleotides and the oligonucleotide having a sequence selected from:

5′ ATCTGGTGAGCCACTG 3′,
5′ AGCTGGTGAGCCACTG 3′,
5′ TGAGCCACTGTTCTTT 3′,
5′ TAAGCCACTGTTCTTT 3′,
5′ GTGAGCCACTGTTCTT 3′,
5′ AACAGTGTAATCTGGT 3′,
5′ AACAGTGTAATCTGAT 3′,
5′ ACAGTGTAATCTGGTG 3′,
5′ GTGTAATCTGGTGAGC 3′,
5′ GTGTAAGCTGGTGAGC 3′,
5′ GTAATCTGGTGAGCCA 3′,
5′ GTAAGCTGGTGAGCCA 3′,
and
5′ AATCTGGTGAGCCACT 3′.

In accordance with another aspect of the invention, there is provided an antisense gapmer oligonucleotide having 5 nucleotide long 5′ and 3′ wings comprising 2′-MOE modified nucleotides and the oligonucleotide having a sequence selected from:

5′ GTGTAATCTGGTGAGCCACT 3′,
5′ GTGTAAGCTG GTGAGCCACT 3′,
5′ CAGTGTAATCTGGTGAGCCA 3′,
and
5′ CAGTGTAAGCTGGTGAGCCA 3′.

In accordance with embodiments, the oligonucleotide of the invention has a backbone that is phosphorothioated.

In accordance with embodiments, the oligonucleotide of the invention comprises 2′-MOE modified nucleotides.

In accordance with embodiments, the oligonucleotide of the invention comprises locked nucleotides.

In accordance with embodiments, the oligonucleotide of the invention comprises a single mismatch.

In accordance with another aspect of the invention, there is provided a pharmaceutical composition comprising the oligonucleotide of the invention or salt thereof and a pharmaceutically acceptable solvent, diluent, carrier, salt or adjuvant, optionally the pharmaceutical composition is formulated for oral or intravenous delivery.

In some embodiments, there is provided a pharmaceutical composition comprising an antisense gapmer oligonucleotide as described herein and a pharmaceutically acceptable salt for use as a therapeutically active substance.

In accordance with another aspect of the invention, there is provided a method of treating a subject, optionally human, having Fibrodysplasia ossificans progressiva (FOP) comprising: administering the oligonucleotide of the invention, or a pharmaceutical composition comprising the same. Optionally, the method comprises a pre-screening step wherein the patient is pre-screened for the 617G>A mutation.

### BRIEF DESCRIPTION OF THE FIGURES

Embodiments of the present invention will now be described, by way of example only, with reference to the attached Figures.

FIG. 1 shows LNA gapmers targeting ACVR1/ALK2 (A) Structures of DNA and LNA. The entire backbone of each gapmer is phosphorothioated. (B) Target sites of the LNA gapmers. NA: non-allele-specific gapmers. AL: allele-specific gapmers. Dark grey 5′ and 3′ UTR. Light grey: coding exons.

FIG. 2 shows that LNA gapmers reduced ACVR1/ALK2 expression in human FOP fibroblasts in vitro. A. Total ACVR1 expression in LNA gapmer-treated fibroblasts in GM00513 human fibroblast cells from female FOP patient measured by qPCR. B. Total ACVR1 expression in LNA gapmer-treated fibroblasts in GM00783 human fibroblast cells from male FOP patient measured by qPCR. The mRNA level of rps18 is used as a control. Repeated measures one-way ANOVA followed by Tukey's post hoc test. ^{***}p<0.005 versus control-treated cells. ^{#}p<0.05, ^{##}p<0.01, ^{###}p<0.005 versus AL-2m. ^{&}p<0.05, &&p<0.01, &&&p<0.005 versus AL-4m. n=3 (biological triplicate). (C) Western blotting of ACVR1 protein in LNA gapmer-treated human FOP fibroblasts (GM00513). (D) Quantification of (C). Expression of ACVR1 relative to GAPDH. Repeated measures one-way ANOVA followed by Dunnett's post hoc test. ^{*}p<0.05, ^{**}p<0.01, ^{***}p<0.005 versus control-treated cells. Means±SD. Expression in the non-treated cells was taken as 100%. The LNA gapmers were transfected at 100 nM. The cells were harvested 24 h after transfection. n=3 (biological triplicate).

FIG. 3 shows LNA gapmers reduced ACVR1^{R206H} expression in human FOP fibroblasts in an allele-specific manner. Relative expression of ACVR1 wild-type and ACVR1^{R206H} alleles measured by allele-specific qPCR in LNA-treated human FOP fibroblasts (A, GM00513; B, GM00783). The LNA gapmers were transfected at 20 nM into the human FOP fibroblasts. Light grey bar: ACVR1 wild-type. Dark grey bar: ACVR1^{R206H}. Expression of each allele in non-treated cells was taken as 100%. The mRNA level of rps18 is used as a control. (C) Western blotting of LNA gapmer-treated C2C12 cells with anti-V5 and GAPDH antibodies. The plasmids expressing ACVR1-V5 or ACVR1^{R206H}-V5 were co-transfected with 1 nM LNA gapmers into C2C12 cells. W: ACVR1-V5. F: ACVR1^{R206H}-V5. (D) Quantification of (C). Relative protein expression of ACVR1 wild-type and ACVR1^{R206H} from the constructs. The expression of each protein in non-treated cells was taken as 100%. GAPDH was used as a control. Light grey bar: ACVR1 wild-type. Dark grey bar: ACVR1^{R206H}. Repeated measures one-way ANOVA followed by Tukey's post hoc test. ^{*}p<0.05; ^{**}p<0.01, ^{***}p<0.005: wild-type versus ACVR1^{R206H} allele. ^{###}p<0.005: versus wild-type ACVR1 expression in control-treated cells. ⁺⁺⁺p<0.005: versus ACVR1^{R206H} expression in control-treated cells. ^{!!!}p<0.005: versus ACVR1^{R206H} expression in AL-6-treated cells. ^{&&&}p<0.005: versus ACVR1^{R206H} expression in AL-7-treated cells. ^{%%%}p<0.005: versus ACVR1^{WT} expression in AL-7-treated cells. NT: treated by the transfection reagent without gapmers. The cells were harvested 24 h after transfection. Means±SD. n=3 (biological triplicate).

FIG. 4 shows allele-specific LNA gapmers efficiently suppress osteogenic marker Alp expression induced by Activin A in ACVR^{R206H} expressing C2C12 myoblasts. The plasmids which encode V5-tagged ACVR^{WT} or ACVR^{R206H} were co-transfected with 5 nM LNA gapmers into C2C12 cells. After 24 h transfection, the cells were cultured for four days with rhActivin A (100 ng/ml). (A) Activin A induces Alp expression in ACVR^{R206H} transfected C2C12 cells. (B, C) LNA gapmers AL-1s and AL-7s repress ALP expression. (A, B) Alkaline phosphatase (ALP) staining. (C) qPCR of Alp gene. Non-treated=100%. Repeated measures one-way ANOVA followed by Dunnett's post hoc test. ^{***}p<0.005 Mean±S.D. n=3 (biological triplicate).

FIG. 5 shows relative expression of ACVR1^{WT} and ACVR1^{R206H} transcripts measured by allele-specific qPCR in 2′MOE gapmer-treated human FOP fibroblasts. The 2′MOE gapmers from Table 3 were transfected into the human FOP fibroblasts. Light grey bar: ACVR1^{WT}. Dark grey bar: ACVR1^{R206H}. Expression of each allele in non-treated cells was taken as 100%. The mRNA level of rps18 is used as a control. ^{*}p<0.05, ^{**}p<0.01, ^{***}p<0.005: wild-type versus ACVR1^{R206H} allele. Two-tailed paired test.

FIG. 6 shows LNA and 2′OMe modified gapmers were equally efficient in vitro. In vitro ACVR1^{R206H} knockdown efficacy of 10 nM and 100 nM doses of MOE3, LNA16, and LNA18 gapmers with and without 2′-OMe modification. Gapmers were lipofected into the cells using 3% Lipofectamine RNAiMAX. The cells were harvested 48 hours post-treatment. Predesigned TaqMan Gene Expression Assays were performed, and the expression of ACVR1 was normalized against RPS18. The qPCR data were then processed using a standard 2^{-ΔΔCT} algorithm and statistically analyzed using one-way ANOVA with posthoc Tukey's comparison test. ^{*}p<0.05; ^{**}p<0.01; ^{****}p<0.0001.

FIG. 7 in vivo systemic injection with LNA 16 and MOE3 reduced the expression of FOP ACVR1. In vivo ACVR1^{R206H} knockdown efficacy of MOE3 and LNA16 in approximately 17-week old, weight-matched (±1 day & ±0.5 gm at day 7) R26-M2rtTA x tetOp-ACVR1(R206H)-IRES-mCHerry mice received ˜100 μL of injection volume containing 7.5 mg/kg (L) or 33 mg/kg (H) gapmers added in phosphate-buffered saline (PBS) once through retro-orbital route. Non-treated group received only PBS. The mice were euthanized 72 hours post-treatment for harvesting tissue samples. Predesigned TaqMan Gene Expression Assays were performed, and the expression of ACVR1 was normalized against RPS18. The qPCR data were then processed using a standard 2^{-ΔΔCT} algorithm and statistically analyzed using one-way ANOVA with posthoc Tukey's comparison test. *p<0.05; **p<0.01; ****p<0.0001.

FIG. 8 shows in vivo systemic injection with LNA16 and MOE3 stalled weight loss and resulted in modest functional improvements. Functional improvements due to MOE3 and LNA 16 treatment of approximately 17 wks old R26-M2rtTA x tetOp-ACVR1(R206H)-IRES-mCHerry mice that received ˜100 μL of injection volume containing 7.5 mg/kg (L) or 33 mg/kg (H) gapmers in phosphate-buffered saline (PBS) or PBS only through retro-orbital route. The body weights of the mice were recorded every day throughout the experimental window. Grip strengths were normalized to body weight. For treadmill running time experiments, the mice were allowed to explore the treadmill for 5 mins before it was turned on and then forced to run to exhaustion over the conveyer belt with gradually increasing speed by 2m/min after running for 5 mins of running at 10m/min. Data were statistically analyzed using one-way ANOVA with posthoc Tukey's comparison test. *p<0.05.

FIG. 9 shows in vivo systemic injection with LNA16 and MOE3 reduced ectopic mineralization in FOP-ACVR1 conditional transgenic mice. Representative 3D reconstructed uCT scans of the head of ˜17 wks old R26-M2rtTA x tetOp-ACVR1(R206H)-IRES-mCHerry mice post-treatment with LNA16 and MOE3 gapmers. The mice received ˜100 μL of injection volume containing 33 mg/kg gapmers added in phosphate-buffered saline (PBS) or PBS only through the retro-orbital route. The non-treated head shows signs of heterotopic ossification outside the skull; treatment with gapmers significantly reduced the presence of these signs.

### DETAILED DESCRIPTION

The invention provides antisense gapmers and compositions comprising gapmers for the treatment of FOP, and methods of treating FOP by allele-specific silencing. Amino acid residue 206 is highly conserved among vertebrates and in human ACVR1 family members. Residue 206 is at the end of the highly conserved glycine/serine (GS) activation domain at the junction of the protein kinase domain. The R206H amino acid substitution is a result of a missense mutation in nucleotide 617 of ACVR1 cDNA (617G>A). More than 95% of FOP is caused by a recurrent mutation of 617G>A. Accordingly, gapmers of the invention are designed to specifically target the mutant allele encoding ACVR1^{R206H} protein preferentially over the allele encoding the wildtype protein and are substantially complementary to a section of mRNA including the 617G>A mutation.

### Oligonucleotides

An antisense gapmer is an oligonucleotide substantially complementary to a target sequence and comprises at least three distinct regions: a 5′-Region (5′R), a central Gap Region (GR), and a 3′ Region (3′R). The 5′R may also be referred to as a 5′ Flank region or a 5′ Wing. The 3′R may also be referred to as a 3′Flank or a 3′Wing.

In one embodiment, the gap region (GR) comprises a sequence complementary to the sequence encoding amino acid residue 206 of the ACVR1^{R206H} protein.

In one embodiment, the 3′ region (3′R), comprises a sequence complementary to the sequence encoding amino acid residue 206 of the ACVR1^{R206H} protein.

In some embodiments, the overall length of the antisense gapmer oligonucleotide is between about 15 and about 25 nucleotides, between about 15 to about 20 nucleotides, 15 to 16 nucleotides, 15 nucleotides, 16 nucleotides, 17 nucleotides, 18 nucleotides, 19 nucleotides, or 20 nucleotides in length.

In one embodiment, the length of the antisense gapmer oligonucleotide is 15 or 16 nucleotides.

In one embodiment, the length of the antisense gapmer oligonucleotide is 20 nucleotides.

In one example, the gap region (GR) is 9 or 10 nucleotides in length with the remaining length made up by approximately equal length 5′ and 3′ flank regions.

Optionally, the 5′-wing region comprises modified nucleotides; the central gap region comprises nucleotides of a different type from the wings, e.g., nucleotides capable of inducing RNase H cleavage; and the 3′-wing region comprises modified nucleotides.

In some embodiments, the 5′-wing region and the 3′-wing region each comprise 2-6 nucleotides, e.g., 2, 3, 4, 5, or 6 nucleotides with one or more of these nucleotides optionally being modified (e.g., 1, 2, 3, 4, 5, or 6 of the nucleotides is modified). The central gap region may comprise 6 or more contiguous DNA nucleosides, linked by phosphodiester or thiophosphate (“ps”) internucleotide linkages. In some embodiments, all internucleotide linkages in the gapmer are thiophospate internucleotide linkages.

In other embodiments, the central gap region includes one or more modified nucleotides. For example, the central region may include one or more modified nucleotides. In some embodiments, the central gap region comprises 6, 7, 8, 9, 10, or 11 contiguous DNA nucleosides. In some embodiments, 1, 2, 3, 4, 5, 6, 7, 8, 9, 10, or 11 of the DNA nucleosides in the central gap region are modified.

In one embodiment, the gap region (GR) consists of a stretch of 9 or 10 contiguous DNA nucleotides.

In some embodiments, the gapmer comprises at least one 2′ sugar modified nucleoside. A 2′ sugar modified nucleoside is a nucleoside which has a substituent other than H or —OH at the 2′ position (2′ substituted nucleoside) or comprises a 2′ linked biradicle and includes 2′ substituted nucleosides and LNA (2′-4′ biradicle bridged) nucleosides. For example, the 2′ modified sugar may provide enhanced binding affinity and/or increased nuclease resistance to the oligonucleotide.

In some embodiments, the modified nucleotides are(S)-constrained ethyl (cEt), 2′-O-methyl, 2′fluoro, ethylene-bridged nucleic acids (ENA), or combinations thereof.

In some embodiments, the 5′ region (5′R) and 3′ region (3′R) each comprise one or more sugar modified nucleotides, such as high affinity sugar modified nucleotides. In some examples, the one or more sugar modified nucleotides in the 5′ region (5′R) and 3′ region (3′R) are 2′-sugar modified nucleotides, such as LNA (Locked nucleic acids) or 2′MOE (2′-O-methoxyethyl-RNA).

In a specific embodiment, 5′ region (5′R) and 3′region (3′R) each consist of five sugar modified nucleotides, which is 2′MOE (2′-O-methoxyethyl-RNA).

In one embodiment, the gap region (GR) comprises 2′OMe (2′-O-methyl) nucleotides, optionally a stretch of 9 or 10 contiguous 2′OMe (2′-O-methyl) nucleotides.

In some embodiments, the gapmer comprises at least one locked nucleic acid (LNA) nucleoside. In a specific example, the 5′ region (5′R) and 3′region (3′R) each consist of three sugar modified nucleotides, which are LNA (Locked nucleic acids).

In some embodiments, the antisense gapmer is 15 to 20 nucleotides in length and consists of linked nucleotides complementary to an equal length portion of exon 6 of ACVR1, the antisense gapmer oligonucleotide have a 5′ region (5′R) comprising three 2′ sugar modified nucleotides, such as LNA or 2′MOE, a central gap region (GR) and a 3′ region (3′R) comprising three 2′ sugar modified nucleotides, such as LNA or 2′MOE, wherein the 5′ region (5′R), gap region (GR), or the 3′ region (3′R), comprises a sequence complementary to a portion of exon 6 of ACVR 1 having the 617G>A mutation of ACVR1 encoding the ACVR1^{R206H}.

In some embodiments, the gapmer is complementary to the ACVR1 mRNA sequence comprising the target sequence 5′-CACCAG-3′, wherein the bolded underlined “A” is the 617G>A mutation.

In some embodiments, the gapmer is complementary to the ACR1 mRNA sequence comprising the target sequence 5′-CACCAGAUU-3′, wherein the bolded underlined “A” is the 617G>A mutation.

In some embodiments, the gapmer is complementary to ACR1 mRNA sequence comprising the target sequence 5′-AACAGUGGCUCACCAGAU-3′, wherein the bolded underlined “A” is the 617G>A mutation.

The term “complementary” means that the antisense oligonucleotide sequence can form hydrogen bonds with the target mRNA sequence by Watson-Crick or other base-pair interactions.

In one embodiment, the 5′ region (5′R), gap region (GR), or the 3′ region (3′R), comprises a sequence complementary to a portion of exon 6 of ACVR1 having the 617G>A mutation of ACVR1 encoding the ACVR1^{R206H}.

In one embodiment, the 5′ region comprises a sequence complementary to a portion of exon 6 of ACVR1 having the 617G>A mutation of ACVR1 encoding the ACVR1^{R206H}.

In one embodiment, the gap region (GR) comprises a sequence complementary to a portion of exon 6 of ACVR1 having the 617G>A mutation of ACVR1 encoding the ACVR1^{R206H}.

In one example, the 3′ region (3′R), comprises a a sequence complementary to a portion of exon 6 of ACVR1 having the 617G>A mutation of ACVR1 encoding the ACVR1^{R206H}.

Specific examples of the antisense gapmer oligonucleotides include:

AL-1
ATCTGGT*GAGCCACTG
AL-1s
AgCTGGT*GAGCCACTG
AL-2
T*GAGCCACTGTTCTTT
AL-2m
T*aAGCCACTGTTCTTT
AL-3
GT*GAGCCACTGTTCTT
AL-4
AACAGTGTAATCTGGT*
AL-4m
AACAGTGTAATCTGaT*
AL-5
ACAGTGTAATCTGGT*G
AL-6
GTGTAATCTGGT*GAGC
AL-6s
GTGTAAgCTGGT*GAGC
AL-7
GTAATCTGGT*GAGCCA
AL-7s
GTAAgCTGGT*GAGCCA
AL-8 AATCTGGT*GAGCCACT
Underlined = LNA
T* = complementary to ACVR1^{R206H} mutation
Lower-case letter = mismatched.

Additional specific examples of the antisense gapmer oligonucleotides include:

FOP 2′MOE_AS1
GTGTAATCTGGT*GAGCCACT
FOP 2′MOE_AS2
GTGTAAgCTG GT*GAGCCACT
FOP 2′MOE_AS3
CAGTGTAATCTGGT*GAGCCA
FOP 2′MOE_AS4
CAGTGTAAgCTGGT*GAGCCA
Underlined= 2′-MOE (2′-O-methoxyethyl-RNA), no mark = DNA, All of the backbones are phosphorothioated.
T* = complementary to ACVR1^{R206H} mutation
Lower-case letter = mismatched

### Methods of Treatment

Fibrodysplasia ossificans progressiva (FOP) is a genetic disorder where typically the systemic connective tissues ossify progressively and heterotopically (heterotopic ossification) and the lowered mobility or the deformation of the limbs and the trunk occurs from the childhood. The heterotopic ossification is an ossification that is observed in a tissue where osteogenesis does not naturally occur. Symptoms of FOP include, but are not limited to, flare-ups and/or heterotopic ossification. Flare-ups refer to the swellings accompanied by inflammation or pain; and the heterotopic ossification refers to the aberrant formation and growth of cartilages or bones. In FOP, the motor function is significantly impaired.

The invention therefore provides methods of treating a subject having Fibrodysplasia ossificans progressiva (FOP) comprising administering a therapeutically effective amount of the one or more oligonucleotides of the invention, or pharmaceutical composition of the invention alone or in combination with other therapeutics.

Optionally, patients suspected of having FOP are screened for the 617G>A mutation.

The invention further provides methods of preventing or limiting heterotopic ossification of subjects identified as having the 617G>A mutation. These methods comprise administering an effective amount of one or more oligonucleotides of the invention, or pharmaceutical composition of the invention prior to heterotopic ossification alone or in combination with other therapeutics.

The invention further provides methods of preventing flare-ups in subjects identified as having the 617G>A mutation. These methods comprise administering an effective amount of the one or more oligonucleotides of the invention, or pharmaceutical composition of the invention prior to a flare-up alone or in combination with other therapeutics.

Gapmers and compositions comprising the same for “treatment” or “use in treating” FOP are provided, wherein the gapmers selective silencing or partial silencing of the mutant allele transcript encoding the ACVR1^{R206H} protein, while substantial sparing the wildtype transcript.

The term “treating” or “treatment” as used herein refers to any process, action, application, therapy, or the like, wherein a subject, including a human being, is subjected to medical intervention with the object of improving the subject's condition, directly or indirectly and/or preventing further decline in the subject's conditions.

In one embodiment, the term “treating” or “treatment” refers to reducing incidence of, preventing incidence of, and/or reducing heterotopic ossification.

The skilled artisan would understand that treatment does not necessarily result in the complete absence or removal of symptoms of FOP.

In other embodiments, the term “treating” or “treatment” includes limiting heterotopic ossification once it has started.

The term “subject”, may refer to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

The term “amelioration” or “ameliorates” as used herein refers to a decrease, reduction or elimination of a condition, disease, disorder, or phenotype, including an abnormality or symptom.

In some embodiments, treatment of FOP is administered prior to the onset of the flare-ups or the heterotopic ossification.

In some embodiments, treatment of FOP is administered avoid or suppress the onset of the flare-ups or heterotopic ossification.

In some embodiments, treatment of FOP is administered when inflammation, pain, flare-ups or the like occurs.

A “therapeutically effective amount” of a compound or composition described herein is an amount sufficient to provide a therapeutic benefit in the treatment of a condition or to delay or minimize one or more symptoms associated with the condition. A therapeutically effective amount of a compound or composition means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

### Pharmaceutical Composition

In a further aspect, the invention provides pharmaceutical compositions comprising one or more of the aforementioned gapmers and/or combination thereof or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution. The invention provides a sodium or potassium salt of the gapmers of the invention.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Gapmers of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration.

The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the pharmaceutical compositions in specifically formulated for oral or intravenous administration.

The terms “physiologically tolerable carriers” and “biocompatible delivery vehicles” are used interchangeably. Thus, the term “carrier” or “excipient” may refer to a non-toxic solid, semi-solid or liquid filler, diluent. The term includes solvents, dispersion, media, coatings, isotonic agents, and adsorption delaying agents, and the like. The use of such media and agents for pharmaceutically active substances is well known in the art.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

The antisense gapmer oligonucleotides, compounds, and compositions, as described herein, may be administered to a subject by any appropriate route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); parenteral, for example, by injection, including intravenous.

Antisense gapmer oligonucleotides, compounds and/or compositions, as described herein may be used in combination with additional/alternate treatments or treatment regimes, as would be known to the skilled worker.

In some examples, drugs to relieve pain and swelling associated with FOP during acute flare-ups (most notably corticosteroids) and non-steroidal anti-inflammatory medication between flare-ups, may be used.

The antisense gapmer oligonucleotides, compounds and/or compositions, as described herein may be administered separately, by the same or different route of administration, or together in the same pharmaceutical composition as the other agents. A skilled worked will be able to determine the appropriate dose for the individual subject. Preparation and dosing schedules for commercially available second therapeutic and other compounds administered in combination with or concomitantly with compounds or compositions described herein may be used according to manufacturers' instructions or determined empirically by the skilled practitioner.

The formulations may conveniently be presented in unit dosage form and may be prepared by any methods well known in the art of pharmacy. Such methods include the step of bringing the antisense gapmer oligonucleotides, compounds and/or compositions, active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The method of the invention may be conveniently practiced by providing the compounds and/or compositions used in such method in the form of a kit. Such kit preferably contains the composition. Such a kit preferably contains instructions for the use thereof.

Unless defined otherwise, all technical and scientific terms used herein have the same meaning as commonly understood by one of ordinary skill in the art to which this invention belongs.

To gain a better understanding of the invention described herein, the following examples are set forth. It should be understood that these examples are for illustrative purposes only. Therefore, they should not limit the scope of this invention in any way.

### EXAMPLES

### Materials and Methods

Cell culture Human FOP fibroblasts (GM00513 and GM00783) were provided by the Coriell NIGMS Human Genetic Cell Repository. Sanger sequencing was used to confirm that both cell lines carry the ACVR1^{R206H} mutation. The cells were maintained in DMEM/F12 media containing 10% fetal bovine serum (FBS; Sigma) and 0.5% penicillin-streptomycin (5000 U/ml, Gibco). Murine C2C12 myoblasts were cultured in DMEM media containing 15% FBS and 0.5% penicillin-streptomycin (5000 U/ml).

### Antisense Oligonucleotides and Transfection

All LNA gapmers were synthesized by Exiqon/QIAGEN. Gapmers were designed to be 15 to 16 nucleotides in length. The first 3 bases and last 3 bases have LNA chemistry, and the backbone is fully phosphorothioated.

Fibroblasts were seeded onto 12-well plates (5×10⁴ cells per well) and cultured for 24 h prior to transfection. The gapmer transfection was performed with 0.3% Lipofectamine RNAiMAX (Life Technologies) for 24 h [23].

C2C12 cells were seeded onto 12-well plates (5×10⁴ cells per well) and cultured for 24 h prior to transfection. For western blotting, the plasmids encoding V5-tagged ACVR1 were co-transfected with 1 nM LNA gapmers using 0.4% Lipofectamine 2000 transfection reagent (Life Technologies). To express approximately the same amount of proteins, 500 ng of V5-ACVR1 and 375 ng of V5-ACVR1^{R206H} were transfected per well, respectively. The V5-tagged ACVR1 was driven by a ubiquitous EF-1α promoter. The V5-tagwas fused to the C-terminus of ACVR1. The cells were harvested 24 h after transfection. For Alp staining, 500 ng of the plasmids with 5 nM LNA gapmers were transfected per well.

### qPCR

Total RNA was extracted using TRIzol Reagent (Life Technologies). cDNA was generated with SuperScript IV Reverse Transcriptase (Life Technologies) as directed by the manufacturer, using oligo (dT)12-18 (Life Technologies). The qPCR reaction was run with a QuantStudio3 real-time PCR system (Applied Biosystems) and TaqMan Fast Advanced Master Mix (Thermo Fisher Scientific) with a protocol of: (1) 95° C. for 20 s and then (2) 40 cycles of 95° C. for 1 s followed by 60° C. for 20 s (for allele-specific ACVR1 qPCR, 63° C. for 20 s). Pre-designed TaqMan Gene Expression assays (Thermo Fisher Scientific) were performed for the qPCR of total ACVR1 (Hs00153836_m1), Alpl (Mm00475834_m1), and rps18 (Hs01375212_g1). Custom TaqMan SNP genotyping assays were used for the allele-specific qPCR of ACVR1 (Assay ID, ANKA3PJ, Thermo Fisher Scientific). The forward primer sequence is CTCTGGTCTTCCTTTTCTGGTACAA. The reverse primer sequence is CCCGACACACTCCAACAGT. The reporter 1 sequence is AGTGGCTCGCCAGATT (VIC). The reporter 2 sequence is CAGTGGCTCACCAGATT (FAM). Expression values were normalized against rps 18 with the ″′1″′1Ct algorithm.

### Western Blotting

Protein was harvested from cells using Pierce RIPA lysis buffer (Thermo Scientific) and loaded on NuPAGE 8% Bis-Tris Midi Gels (Life Technologies). Primary antibodies used were the rabbit monoclonal anti-activin receptor type I antibody (diluted 1:4000, ab155981, Abcam), the rabbit monoclonal anti-GAPDH antibody (diluted 1:10000, #2118, Cell Signaling Technology), and the mouse monoclonal anti-V5 antibody (diluted 1:5000, R960-25, Invitrogen). Secondary antibodies used were HRP-conjugated goat anti-mouse IgG (H+L) (Bio-Rad) and HRP-conjugated goat anti-rabbit IgG (H+L) (Bio-Rad), both diluted 1:10000. Bands were detected using the Amersham ECL Select Western Blotting Detection kit (GE Healthcare) and the ChemDoc Touch Imaging System (Bio-Rad). The intensity of bands was quantified with Image Lab Software (Bio-Rad). Alp staining

After 24 h transfection, the cells were cultured for four days with recombinant human activin A (rhActivin A) (100 ng/ml, R&D Systems). Cells were fixed with 4% paraformaldehyde and stained by Alkaline Phosphatase Detection Kit (EMD Millipore) according to the manufacturer's instruction.

### Results

### LNA Gapmers Effectively Knock Down ACVR1/ALK2 Expression In Vitro

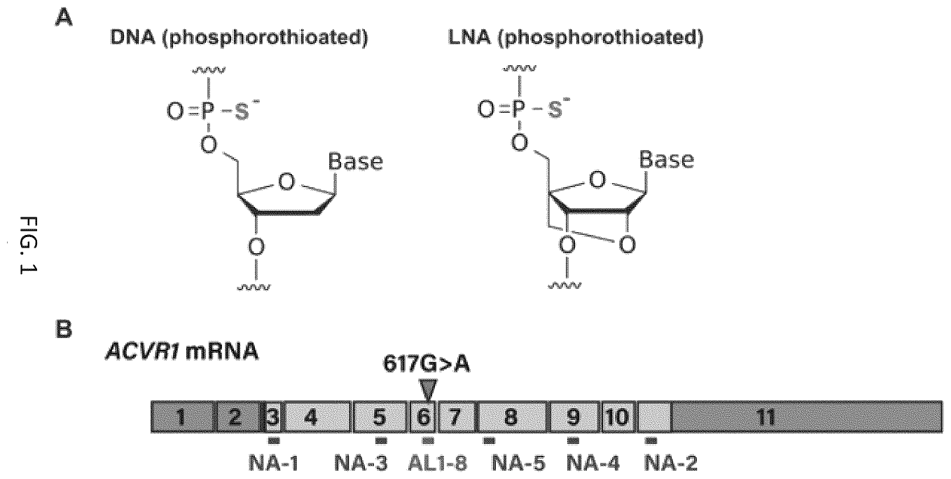

To reduce ACVR1^{R206H} activity, LNA gapmers against ACVR1 (FIG. 1) were designed. LNAs are modified RNA nucleotides that contain a methylene bridge connecting the 2′-O with the 4′-C position in the furanose ring (FIG. 1A) [25, 26]. The entire backbone is phosphorothioated to increase stability and to enhance protein binding, which is critical to cell uptake and intracellular distribution[27]. The central core of each gapmer has 9 or 10 DNA nucleotides, flanked by 3 LNA nucleotides at both ends. To test whether the region containing the ACVR1^{R206H} mutation (617G>A) is suitable for targeting by gapmers, two types of LNA gapmers were designed: non-allele-specific and allele-specific. The former binds ACVR1 mRNA in a non-allele-specific manner (FIG. 1B, Table 1, LNA gapmers NA-1 to NA-5), while the latter contains the ACVR1^{R206H} mutation (617G>A) on exon 6 (FIG. 1B, Table 2, AL-1 to AL-8).

TABLE 1
LNA gapmers against ACVR1/ALK2

| ID | Sequence (5′-3′) | Length (bp) | location |
|---|---|---|---|
| control | AACACGTCTATACGC | 15 | |
| NA-1 | AGCAATCATGATAAGC | 16 | Exon 3 37-52 |
| NA-2 | GTCTTGCGGATGGATT | 16 | Exon 11 46-51 |
| NA-3 | GGTTGCGCCTTTTAA | 15 | Exon 5 115-129 |
| NA-4 | TTCATCTAGAACTTCG | 16 | Exon 9 86-102 |
| NA-5 | TACTGGAGTGTCTTG | 15 | Exon 8 25-39 |

Underlined = LNA

TABLE 2
Allele-specific locked nucleic acid (LNA) gapmers against mutant ACVR1^{R206H}

| | | Length | | |
|---|---|---|---|---|
| | | (base | Location | |
| ID | Sequence (5′-3′) | pairs) | (Exon 6) | |
| AL-1 | ATCTGGT*GAGCCACTG | 16 | 65-80 | siRNA mutation |
| AL-1s | AgCTGGT*GAGCCACTG | 16 | 65-80 | |
| AL-2 | T*GAGCCACTGTTCTTT | 16 | 59-74 | mismatched |
| AL-2m | T*aAGCCACTGTTCTTT | 16 | 59-74 | |
| AL-3 | GT*GAGCCACTGTTCTT | 16 | 60-75 | |
| AL-4 | AACAGTGTAATCTGGT* | 16 | 74-89 | mismatched |
| AL-4m | AACAGTGTAATCTGaT* | 16 | 74-89 | |
| AL-5 | ACAGTGTAATCTGGT*G | 16 | 73-88 | |
| AL-6 | GTGTAATCTGGT*GAGC | 16 | 70-85 | siRNA mutation |
| AL-6s | GTGTAAgCTGGT*GAGC | 16 | 70-85 | |
| AL-7 | GTAATCTGGT*GAGCCA | 16 | 68-83 | siRNA mutation |
| AL-7s | GTAAgCTGGT*GAGCCA | 16 | 68-83 | |
| AL-8 | AATCTGGT*GAGCCACT | 16 | 66-81 | |

Underlined = LNA
T* = complementary to ACVR1^{R206H} mutation
Lower-case letter = mismatched.

TABLE 3
Allele-specific 2′MOE gapmers against mutant ACVR1^{R206H}

| ID | Sequence (5′-3′) | Location (Exon 6) |
|---|---|---|
| FOP 2′MOE_AS1 | GTGTAATCTGGT*GAGCCACT | 66-86 |
| FOP 2′MOE_AS2 | GTGTAAgCTG GT*GAGCCACT | 66-86 |
| FOP 2′MOE_AS3 | CAGTGTAATCTGGT*GAGCCA | 68-88 |
| FOP 2′MOE_AS4 | CAGTGTAAgCTGGT*GAGCCA | 68-88 |

Underlined=2′-MOE, no mark = DNA, All of the backbones are phosphorothioated.
T* = complementary to ACVR1^{R206H} mutation
Lower-case letter = mismatched

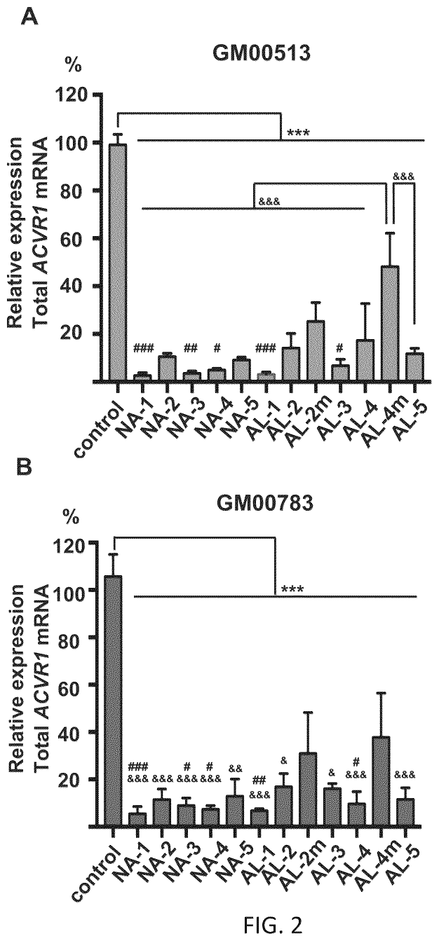

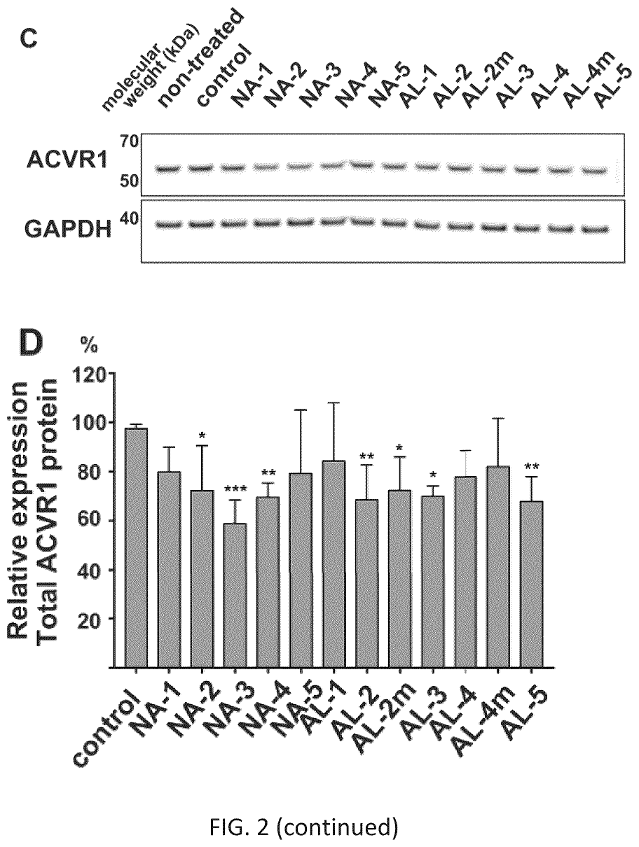

First, the efficacy of the LNA gapmers was evaluated by measuring to ACVR1 expression, including both wild-type and ACVR1^{R206H} allele (FIG. 2A, 2B). Each gapmer was transfected at 100 nM into male and female human FOP fibroblast cell lines (GM00513, GM00783) carrying the ACVR1^{R206H} mutation. Analysis with qPCR revealed that all the LNA gapmers significantly reduced the amount of total ACVR1 mRNA in both lines of human FOP fibroblasts (FIG. 2A, 2B). Also, western blotting revealed that several gapmers successfully reduced ACVR1 protein expression (FIG. 2C, 2D). However, ACVR1 protein levels were not affected as much as ACVR1 RNA, possibly because the ACVR1 protein turnover rate is slower than the mRNA. Overall, these results indicate that the LNA gapmers can reduce the expression of ACVR1 in FOP cells. Furthermore, the region containing the ACVR1^{R206H} mutation is a promising target for gapmers as several ACVR1^{R206H} mutation-targeting gapmers reduced ACVR1 expression significantly, similar to non-allele-specific gapmers.

To determine the specificity of the gapmers to the target sequence, one mismatch was introduced into AL-2 and AL-4 (guanine to adenine, AL-2m, and AL-4m in Table 2) and examined if the single base mismatch can significantly reduce the activity against ACVR1. Both mismatches are located in the ‘LNA wings’ of gapmers. Both single mismatches were expected to decrease the binding ability of the gapmers to the targeted mRNAs because LNA has a higher binding affinity against complementary RNA[22]. The qPCR results demonstrated that the mutation in AL-4 significantly reduced the efficacy of the gapmer (AL-4 versus AL-4m). However, the mutation in AL-2 did not affect the efficacy of the gapmer (AL-2 versus AL-2m) (FIG. 2A, 2B).

### LNA Gapmers Effectively Knock Down ACVR1/ALK2^{R206H} Expression While Leaving Most of the Normal Allele Products Intact In Vitro

Since the R206H mutation (617G>A) in ACVR1 causes FOP, one promising therapeutic strategy is to knock down the mutant transcript by LNA gapmers in an allele-specific manner. Several new allele-specific gapmers (AL-6, 7, and 8) were designed. AL-2, AL-2m, AL-4, and AL-4m were excluded because the R206H mutation is located at the 5′ or 3′ end called ‘LNA wings’; therefore, the allele-specific binding affinity is expected to be low. To enhance the allele specificity, a mismatch (T>G) that improved the allele-specificity in siRNAs targeting the mRNA from the ACVR1^{R206H} allele in a previous study (AL-1s, AL-6s, and AL-7s, Table 2) was introduced.

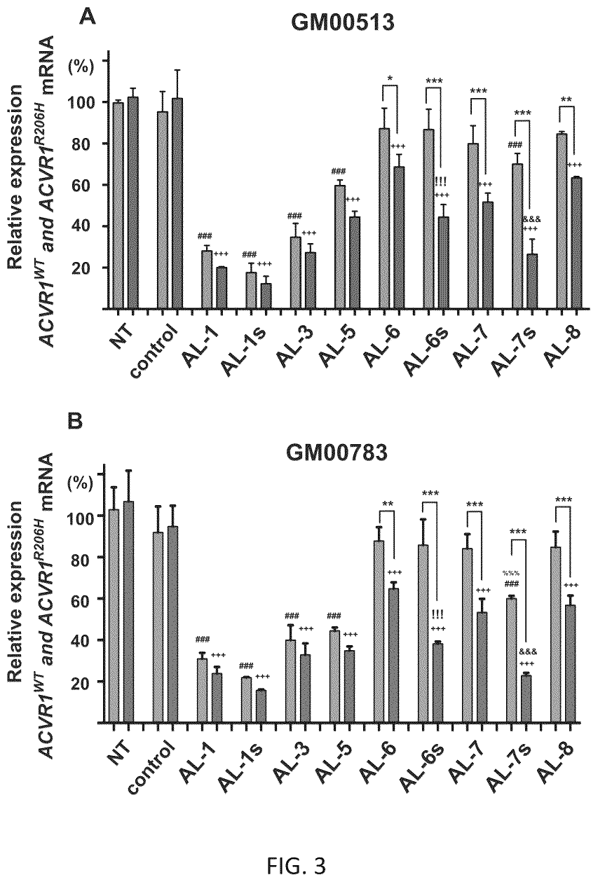

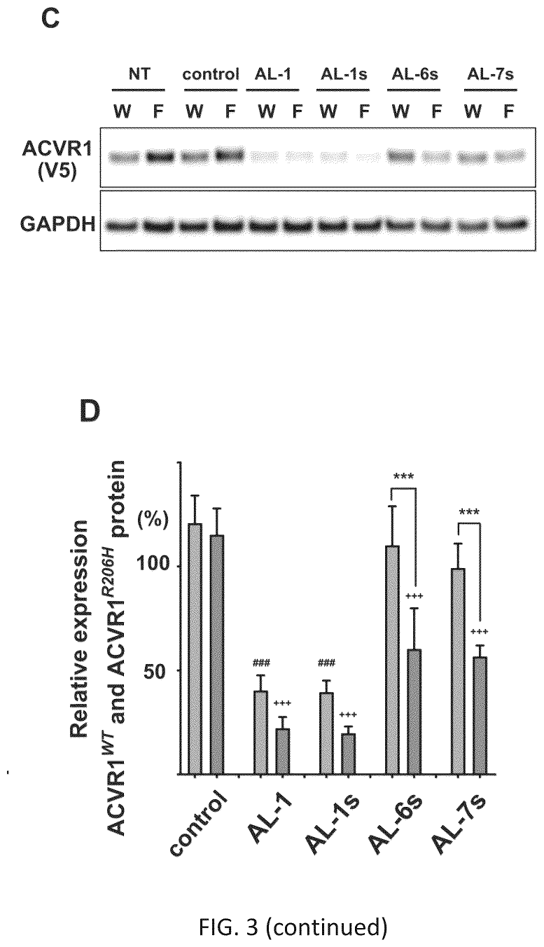

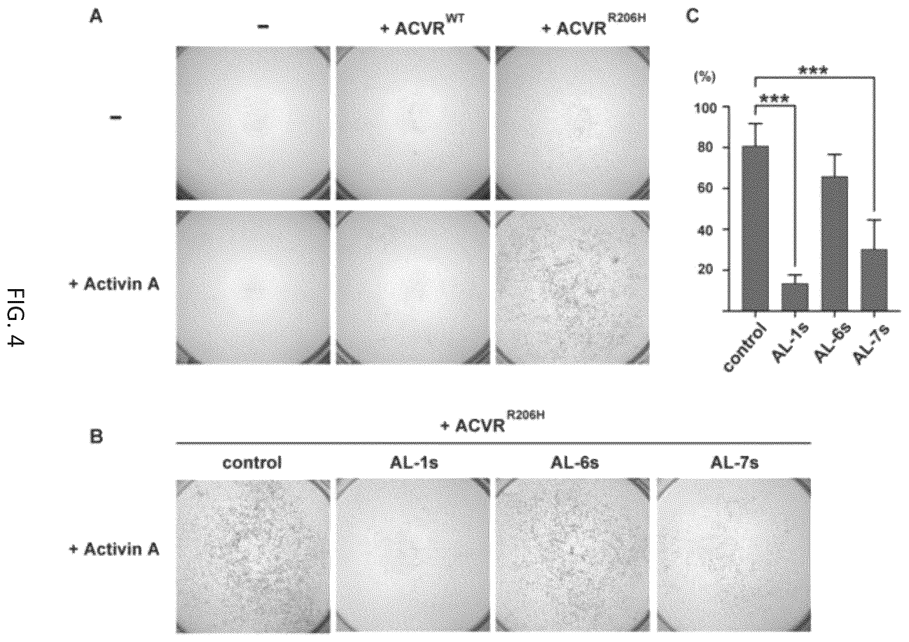

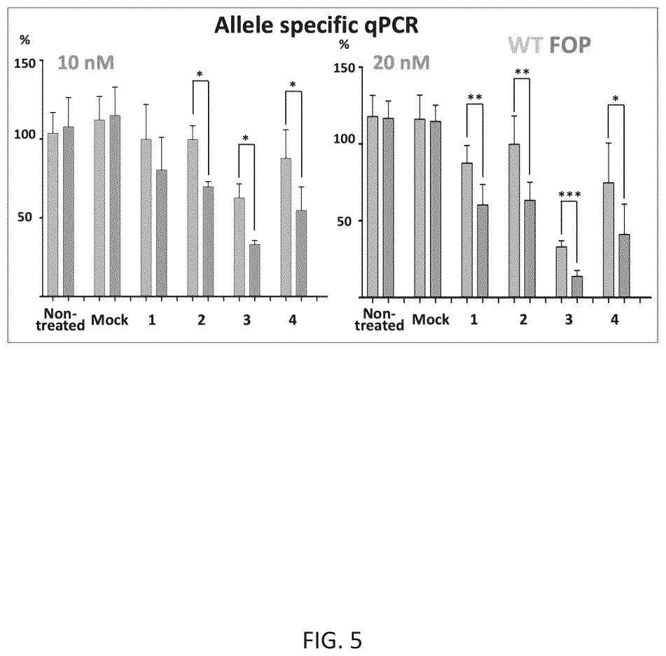

To examine whether the gapmers could knock down ACVR1 mRNA with the 617G>A mutation in an allele-specific manner, the gapmers were transfected into the human FOP fibroblasts and analyzed ACVR1 mRNA expression by allele-specific qPCR. AL-6, AL-6s, AL-7, and AL-8 significantly reduced the mRNAs with the 617G>A mutation without significantly affecting the expression of the ACVR1 wild-type allele (FIG. 3A, 3B). Also, the mismatch enhanced the allele specificity in AL-6s (FIG. 3A, 3B). Meanwhile, AL-7s reduced the expression of ACVR1^{R206H} mRNA significantly more than the expression of ACVR1^{WT}; however, wild-type allele expression was also decreased by AL-7s (FIG. 3A, 3B). On the other hand, allele-specific siRNAs targeting the FOP mutation did not significantly reduce the expression of either ACVR1^{R206H} or ACVR1^{WT} in the FOP patient fibroblasts [16, 17].

To determine whether the gapmers could knock down ACVR1R206H allele-specifically at the protein level, the efficacy of the gapmers in C2C12 cells expressing V5-tagged ACVR1 or ACVR1^{R206H} was analyzed. Western blotting showed that AL-6s and AL-7s significantly reduced the ACVR1^{R206H} protein expression without significantly affecting ACVR1WT (FIG. 3C, 3D). These results indicate that the mismatched LNA gapmers can efficiently knockdown ACVR1^{R206H} expression while leaving most of the normal allele products intact in vitro.

### LNA Gapmers Suppress Osteogenic Differentiation Induced by ACVR1^{R206H} and Activin A

Activin A treatment induces osteogenic differentiation in mesenchymal stromal cells derived from FOP patients' induced pluripotent stem cells (iPSCs)[5], and BMP-2, 4, 6, and 7 treatments induces alkaline phosphatase expression (Alp, an osteogenic differentiation marker) in ACVR1^{R206H}-transfected C2C12 cells [28, 29]. As such, to test whether activin A treatment induces Alp expression in ACVR1^{R206H}-transfected C2C12 cells, ACVR1^{WT} and ACVR1^{R206H} was transfected into C2C12 cells and treated them with rhActivin A for four days. Alp staining revealed that only ACVR1^{R206H}-transfected cells respond to activin A and express Alp after activin A treatment (FIG. 4A).

Next, to analyze whether the gapmers suppress Alp expression, ACVR1^{R206H} and the allele-specific gapmers were co-transfected into C2C12 cells and treated with activin A (FIG. 4B). Alp staining and qPCR demonstrated that AL-1s and AL-7s prevented Alp expression in the C2C12 cells, but AL-6s did not (FIG. 4B, C). These data indicate that allele-specific gapmers AL-1s and AL-7s suppress osteogenic differentiation induced by activin A and ACVR1^{R206H} overexpression.

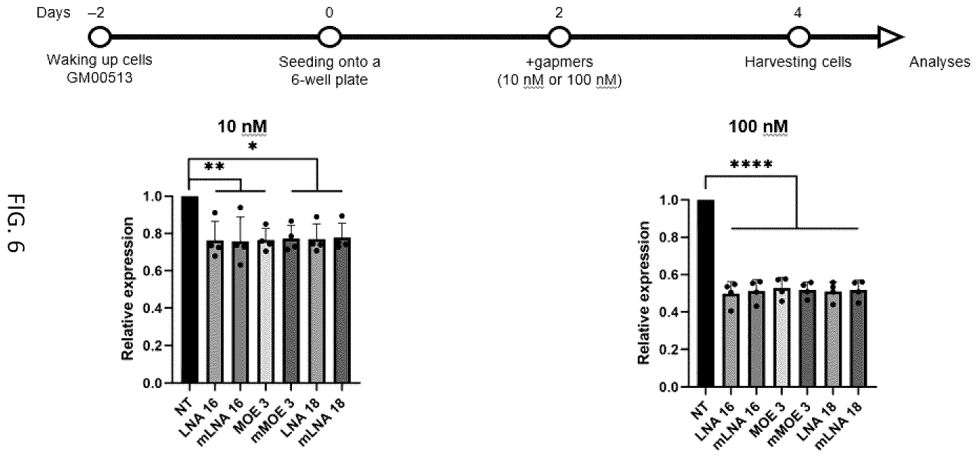

The efficiency of LNA and 2′OMe modified gapmers was compared in vitro and referring to FIG. 6 found to be equally efficient.

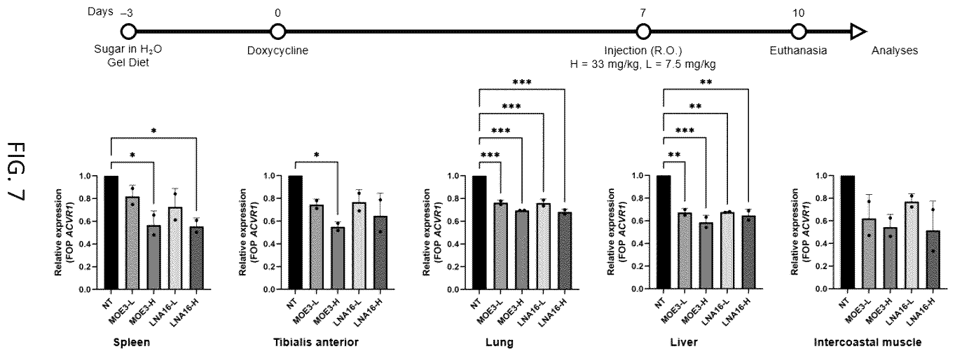

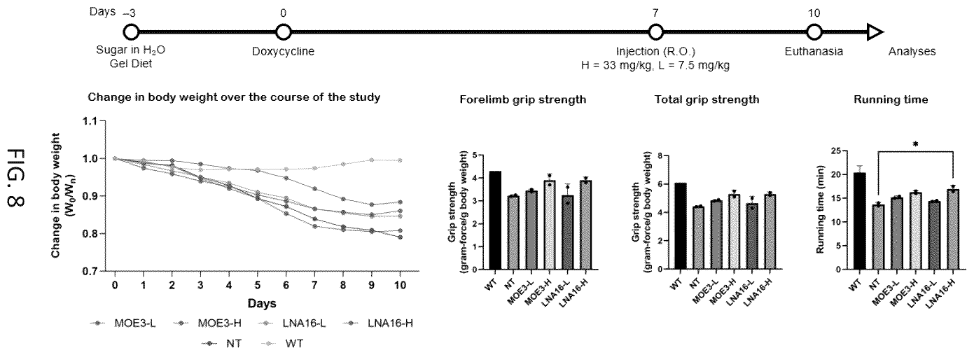

Activity of LNA 16 and MOE3 having the sequences MOE3: 5′-CAG TGT AAT CTG GTG AAG CCA-3′ and LNA16: 5′-AGC TGG TGA GCC ACT G-3′where Italicized: MOE; Underlined: LNA) were assessed in vivo by systemic injection. Referring to FIG. 7 in vivo systemic injection with LNA 16 and MOE3 reduced the expression of FOP ACVR1. Referring to FIG. 8 in vivo systemic injection with LNA 16 and MOE3 stalled weight loss and resulted in modest functional improvements.

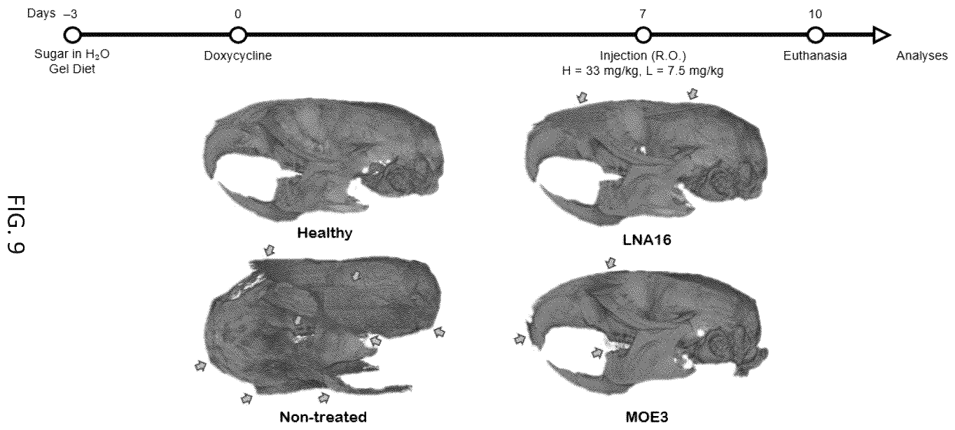

Referring to FIG. 9 shows in vivo systemic injection with LNA16 and MOE3 reduced ectopic mineralization in FOP-ACVR1 conditional transgenic mice. Representative 3D reconstructed micro-CT scans of the head of ˜17 wks old R26-M2rtTA x tetOp-ACVR1(R206H)-IRES-mCHerry mice post-treatment with LNA 16 and MOE3 gapmers shows treatment with gapmers significantly reduced heterotopic ossification outside the skull.

### Discussion

More than 95% of FOP cases are caused by a recurrent mutation ACVR1^{R206H} (617G>A), which is autosomal dominant [2, 3]. A major hurdle for successful therapy in FOP is that deactivation of normal ACVR1 function can have adverse effects. In this study, it is shown that newly-designed LNA gapmers can preferentially reduce ACVR1^{R206H} expression levels of both RNA and protein without affecting wild-type ACVR1. Also, several allele-specific gapmers suppressed osteogenic differentiation induced by activin A and ACVR1^{R206H} overexpression.

The example demonstrates that oligonucleotide-based therapy is an effective strategy to achieve allele-specific knockdown of ACVR1. Studies have already demonstrated the feasibility of using artificial oligonucleotides to reduce ACVR1 expression in vitro. (1) Phosphorodiamidate morpholino oligomer targeting mouse Acvr1 successfully reduces RNA expression by inducing exon 8 skipping (equivalent to human ACVR1 exon 6) and repress BMP6-induced osteoblast differentiation [30]. However, this knock-down is not allele-specific. (2) siRNAs targeting the R206H mutation preferentially suppress the expression of ACVR1 with the mutation 617G>A in the primary dental pulp of exfoliated deciduous teeth (SHED) cells from people with FOP [16]. Furthermore, Takahashi et al. demonstrated that a mismatch in the siRNA improves the allele specificity [17]. In this study, it is demonstrated that LNA gapmers can also knock down ACVR1^{R206H} in an allele-specific manner and that the mismatch enhances the allele-specificity of the LNA gapmer AL-6 (FIG. 3A, 3B). However, the mismatch effect depends on gapmer sequences. The mismatch does not enhance the allele specificity of AL-7 but does augment the knock-down effect of AL-7 against both wild-type and 617G>A allele (FIG. 3A, 3B).

None of the gapmers in this study appear to show toxicity to the cell lines used, but toxicity should be reevaluated in vivo before clinical trials. Interestingly, a recent study demonstrated that introduction of a single 2′-O-methyl modification at gap position 2 reduced hepatotoxicity of toxic LNA gapmers with minimal impairment of antisense activity [38].

The major advantage of antisense oligonucleotide-based drugs is that they can be injected under the skin or directly into a vein. This is particularly beneficial for people with FOP, because gene therapy mediated by virus vectors is likely to lead to immune activation and inflammation, thereby inducing more heterotopic ossification. Also, in vivo delivery of siRNAs requires drug delivery systems such as lipid nanoparticles and GalNAc conjugation [39]. However, although gapmers are effective in vivo without carriers, efficient in vivo delivery systems can further mitigate potential toxicity.

### References

1. Pignolo, R. J., Shore, E. M. and Kaplan, F. S. (2011) Fibrodysplasia ossificans progressiva: clinical and genetic aspects. Orphanet J Rare Dis 6, 80.
2. Shore, E. M., Xu, M., Feldman, G. J., Fenstermacher, D. A., Cho, T. J., Choi, I. H., Connor, J. M., Delai, P., Glaser, D. L. et al. (2006) A recurrent mutation in the BMP type I receptor ACVR1 causes inherited and sporadic fibrodysplasia ossificans progressiva. Nat Genet 38 (5), 525-7.
3. Huning, I. and Gillessen-Kaesbach, G. (2014) Fibrodysplasia ossificans progressiva: clinical course, genetic mutations and genotype-phenotype correlation. Mol Syndromol 5 (5), 201-11.
4. Hatsell, S. J., Idone, V., Wolken, D. M., Huang, L., Kim, H. J., Wang, L., Wen, X., Nannuru, K. C., Jimenez, J. et al. (2015) ACVR1R206H receptor mutation causes fibrodysplasia ossificans progressiva by imparting responsiveness to activin A. Sci Transl Med 7 (303), 303ra137.
5. Hino, K., Ikeya, M., Horigome, K., Matsumoto, Y., Ebise, H., Nishio, M., Sekiguchi, K., Shibata, M., Nagata, S. et al. (2015) Neofunction of ACVR1 in fibrodysplasia ossificans progressiva. Proc Natl Acad Sci U S A 112 (50), 15438-43.
6. Dey, D., Bagarova, J., Hatsell, S. J., Armstrong, K. A., Huang, L., Ermann, J., Vonner, A. J., Shen, Y., Mohedas, A. H. et al. (2016) Two tissue-resident progenitor lineages drive distinct phenotypes of heterotopic ossification. Sci Transl Med 8 (366), 366ra163.
7. Lees-Shepard, J. B., Yamamoto, M., Biswas, A. A., Stoessel, S. J., Nicholas, S. E., Cogswell, C. A., Devarakonda, P. M., Schneider, M. J., Jr., Cummins, S. M. et al. (2018) Activin-dependent signaling in fibro/adipogenic progenitors causes fibrodysplasia ossificans progressiva. Nat Commun 9 (1), 471.
8. Chakkalakal, S. A., Uchibe, K., Convente, M. R., Zhang, D., Economides, A. N., Kaplan, F. S., Pacifici, M., Iwamoto, M. and Shore, E. M. (2016) Palovarotene Inhibits Heterotopic Ossification and Maintains Limb Mobility and Growth in Mice With the Human ACVR1(R206H) Fibrodysplasia Ossificans Progressiva (FOP) Mutation. J Bone Miner Res 31 (9), 1666-75.
9. Hino, K., Horigome, K., Nishio, M., Komura, S., Nagata, S., Zhao, C., Jin, Y., Kawakami, K., Yamada, Y. et al. (2017) Activin-A enhances mTOR signaling to promote aberrant chondrogenesis in fibrodysplasia ossificans progressiva. J Clin Invest 127 (9), 3339-3352.
10. Hino, K., Zhao, C., Horigome, K., Nishio, M., Okanishi, Y., Nagata, S., Komura, S., Yamada, Y., Toguchida, J. et al. (2018) An mTOR Signaling Modulator Suppressed Heterotopic Ossification of Fibrodysplasia Ossificans Progressiva. Stem Cell Reports 11 (5), 1106-1119.
11. Vanhoutte, F., Liang, S., Ruddy, M., Zhao, A., Drewery, T., Wang, Y., DelGizzi, R., Forleo-Neto, E., Rajadhyaksha, M. et al. (2020) Pharmacokinetics and Pharmacodynamics of Garetosmab (Anti-Activin A): Results From a First-in-Human Phase 1 Study. J Clin Pharmacol 60 (11), 1424-1431.
12. Gu, Z., Reynolds, E. M., Song, J., Lei, H., Feijen, A., Yu, L., He, W., MacLaughlin, D. T., van den Eijnden-van Raaij, J. et al. (1999) The type I serine/threonine kinase receptor ActRIA (ALK2) is required for gastrulation of the mouse embryo. Development 126 (11), 2551-61.
13. Mishina, Y., Crombie, R., Bradley, A. and Behringer, R. R. (1999) Multiple roles for activin-like kinase-2 signaling during mouse embryogenesis. Dev Biol 213 (2), 314-26.
14. Dudas, M., Sridurongrit, S., Nagy, A., Okazaki, K. and Kaartinen, V. (2004) Craniofacial defects in mice lacking BMP type I receptor Alk2 in neural crest cells. Mech Dev 121 (2), 173-82.
15. Lees-Shepard, J. B., Nicholas, S. E., Stoessel, S. J., Devarakonda, P. M., Schneider, M. J., Yamamoto, M. and Goldhamer, D. J. (2018) Palovarotene reduces heterotopic in juvenile FOP mice but exhibits pronounced skeletal toxicity. Elife 7, e40814.
16. Kaplan, J., Kaplan, F. S. and Shore, E. M. (2012) Restoration of normal BMP signaling levels and osteogenic differentiation in FOP mesenchymal progenitor cells by mutant allele-specific targeting. Gene Ther 19 (7), 786-90.
17. Takahashi, M., Katagiri, T., Furuya, H. and Hohjoh, H. (2012) Disease-causing allele-specific silencing against the ALK2 mutants, R206H and G356D, in fibrodysplasia ossificans progressiva. Gene Ther 19 (7), 781-5.
18. Skotte, N. H., Southwell, A. L., Ostergaard, M. E., Carroll, J. B., Warby, S. C., Doty, C. N., Petoukhov, E., Vaid, K., Kordasiewicz, H. et al. (2014) Allele-specific suppression of mutant huntingtin using antisense oligonucleotides: providing a therapeutic option for all Huntington disease patients. PLoS One 9 (9), e107434.
19. Southwell, A. L., Skotte, N. H., Kordasiewicz, H. B., Ostergaard, M. E., Watt, A. T., Carroll, J. B., Doty, C. N., Villanueva, E. B., Petoukhov, E. et al. (2014) In vivo evaluation of candidate allele-specific mutant huntingtin gene silencing antisense oligonucleotides. Mol Ther 22 (12), 2093-106.
20. Southwell, A. L., Kordasiewicz, H. B., Langbehn, D., Skotte, N. H., Parsons, M. P., Villanueva, E. B., Caron, N. S., Ostergaard, M. E., Anderson, L. M. et al. (2018) Huntingtin suppression restores cognitive function in a mouse model of Huntington's disease. Sci Transl Med 10 (461).
21. Scoles, D. R., Minikel, E. V. and Pulst, S. M. (2019) Antisense oligonucleotides: A primer. Neurol Genet 5 (2), e323.
22. Wahlestedt, C., Salmi, P., Good, L., Kela, J., Johnsson, T., Hokfelt, T., Broberger, C., Porreca, F., Lai, J. et al. (2000) Potent and nontoxic antisense oligonucleotides containing locked nucleic acids. Proc Natl Acad Sci U S A 97 (10), 5633-8.
23. Maruyama, R., Touznik, A. and Yokota, T. (2018) Evaluation of Exon Inclusion Induced by Splice Switching Antisense Oligonucleotides in SMA Patient Fibroblasts. J Vis Exp (135), (135):57530.
24. Fukuda, T., Kanomata, K., Nojima, J., Kokabu, S., Akita, M., Ikebuchi, K., 10 Jimi, E., Komori, T., Maruki, Y. et al. (2008) A unique mutation of ALK2, G356D, found in a patient with fibrodysplasia ossificans progressiva is a moderately activated BMP type I receptor. Biochem Biophys Res Commun 377 (3), 905-9.
25. Obika, S., Nanbu, D., Hari, Y., Morio, K., In, Y., Ishida, T. and Imanishi, T. (1997) Synthesis of 2′-O,4′-C-methyleneuridine and-cytidine. Novel bicyclic nucleosides having a fixed C-3,-endo sugar puckering. Tetrahedron Letters 38 (50), 8735-8738.
26. Singh, S. K., Nielsen, P., Koshkin, A. A. and Wengel, J. (1998) LNA (locked nucleic acids): synthesis and high-affinity nucleic acid recognition. Chemical Communications (4), 455-456.
27. Crooke, S. T., Wang, S., Vickers, T. A., Shen, W. and Liang, X. H. (2017) Cellular uptake and trafficking of antisense oligonucleotides. Nat Biotechnol 35 (3), 230-237.
28. Fukuda, T., Kohda, M., Kanomata, K., Nojima, J., Nakamura, A., Kamizono, J., Noguchi, Y., Iwakiri, K., Kondo, T. et al. (2009) Constitutively activated ALK2 and increased SMAD1/5 cooperatively induce bone morphogenetic protein signaling in fibrodysplasia ossificans progressiva. J Biol Chem 284 (11), 7149-56.
29. Song, G. A., Kim, H. J., Woo, K. M., Baek, J. H., Kim, G. S., Choi, J. Y. and Ryoo, H. M. (2010) Molecular consequences of the ACVR1(R206H) mutation of fibrodysplasia ossificans progressiva. J Biol Chem 285 (29), 22542-53.
30. Shi, S., Cai, J., de Gorter, D. J., Sanchez-Duffhues, G., Kemaladewi, D. U., Hoogaars, W. M., Aartsma-Rus, A., t Hoen, P. A. and ten Dijke, P. (2013) Antisense-oligonucleotide mediated exon skipping in activin-receptor-like kinase 2: inhibiting the receptor that is overactive in fibrodysplasia ossificans progressiva. PLoS One 8 (7), e69096.
31. Aslesh, T. and Yokota, T. (2020) Development of Antisense Oligonucleotide Gapmers for the Treatment of Dyslipidemia and Lipodystrophy. Methods Mol Biol 2176, 69-85.
32. Mahfouz, M., Maruyama, R. and Yokota, T. (2020) Inotersen for the Treatment of Hereditary Transthyretin Amyloidosis. Methods Mol Biol 2176, 87-98.
33. Chan, L. and Yokota, T. (2020) Development and Clinical Applications of Antisense Oligonucleotide Gapmers. Methods Mol Biol 2176, 21-47.
34. Rupaimoole, R. and Slack, F. J. (2017) MicroRNA therapeutics: towards a new era for the management of cancer and other diseases. Nat Rev Drug Discov 16 (3), 203-222.
35. Carroll, J. B., Warby, S. C., Southwell, A. L., Doty, C. N., Greenlee, S., Skotte, N., Hung, G., Bennett, C. F., Freier, S. M. et al. (2011) Potent and selective antisense oligonucleotides targeting single-nucleotide polymorphisms in the Huntington disease gene/allele-specific silencing of mutant huntingtin. Mol Ther 19 (12), 2178-85.
36. Pfeiffer, N., Voykov, B., Renieri, G., Bell, K., Richter, P., Weigel, M., Thieme, H., Wilhelm, B., Lorenz, K. et al. (2017) First-in-human phase I study of ISTH0036, an antisense oligonucleotide selectively targeting transforming growth factor beta 2 (TGF-beta2), in subjects with open-angle glaucoma undergoing glaucoma filtration surgery. PLoS One 12 (11), e0188899.
37. Frazier, K. S. (2015) Antisense oligonucleotide therapies: the promise and the challenges from a toxicologic pathologist's perspective. Toxicol Pathol 43 (1), 78-89
38. Shen, W., De Hoyos, C. L., Migawa, M. T., Vickers, T. A., Sun, H., Low, A., Bell, T. A., III, Rahdar, M., Mukhopadhyay, S. et al. (2019) Chemical modification of PS-ASO therapeutics reduces cellular protein-binding and improves the therapeutic index. Nat Biotechnol 37 (6), 640-650.
39. Khvorova, A. and Watts, J. K. (2017) The chemical evolution of oligonucleotide therapies of clinical utility. Nat Biotechnol 35 (3), 238-248.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A modified antisense oligonucleotide consisting of 12 to 30 linked nucleotides substantially complementary to a portion of exon 6 of an ACVR1 mutant allele encoding ACVR1^{R206H} mutant protein and comprising a wing-gap-wing motif with a 5′ wing region positioned at the 5′ end of a deoxynucleoside gap region, and a 3′ wing region positioned at the 3′ end of the deoxynucleoside gap region, wherein the 5′ wing region comprises at least three 2′ sugar modified nucleotides, the 3′ wing region comprises at least three 2′ sugar modified nucleotides and wherein the deoxynucleoside gap region comprises a sequence complementary to a portion of exon 6 of ACVR 1 having the 617G>A mutation of the ACVR 1 mutant allele, wherein the gap region comprises the sequence 5′-CTGGTG-3′ and wherein the oligonucleotide optionally comprises phosphorothioate linkages.

2. (canceled)

3. The oligonucleotide of claim 1, comprising the sequence 5′-AATCTGGTG-3′.

4. The oligonucleotide of claim 1, wherein the 2′ sugar modified nucleotides comprises a 2′-O-alkyl substituent or wherein the 2′ sugar modified nucleotide is a locked nucleotide.

5. The oligonucleotide of claim 4, wherein the 2′-O-alkyl substituent is 2′-O-methoxyethyl.

6. (canceled)

7. (canceled)

8. The oligonucleotide of claim 1, wherein the oligonucleotide is 100% complementary to the portion of exon 6 of the ACVR1 mutant allele encoding ACVR1^{R206H} mutant protein over its entire length.

9. The oligonucleotide of claim 1, comprising a mismatch.

10. The oligonucleotide of claim 9, wherein the mismatch is in the 5′ wing region or the 3′ wing region, optionally wherein the mismatch is 5 bases towards the 5′ end from the single nucleotide that anneals to the single nucleotide mutation.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. An antisense gapmer oligonucleotide having 3 nucleotide long 5′ and 3′ wings comprising locked nucleotides and the oligonucleotide having a sequence selected from:
5′ ATCTGGTGAGCCACTG 3′,
5′ AGCTGGTGAGCCACTG 3′,
5′ TGAGCCACTGTTCTTT 3′,
5′ TAAGCCACTGTTCTTT 3′,
5′ GTGAGCCACTGTTCTT 3′,
5′ AACAGTGTAATCTGGT 3′,
5′ AACAGTGTAATCTGAT 3′,
5′ ACAGTGTAATCTGGTG 3′,
5′ GTGTAATCTGGTGAGC 3′,
5′ GTGTAAGCTGGTGAGC 3′,
5′ GTAATCTGGTGAGCCA 3′,
5′ GTAAGCTGGTGAGCCA 3′,
and
5′ AATCTGGTGAGCCACT 3′.

26. An antisense gapmer oligonucleotide having 5 nucleotide long 5′ and 3′ wings comprising 2′-MOE modified nucleotides and the oligonucleotide having a sequence selected from:
5′ GTGTAATCTGGTGAGCCACT 3′,
5′ GTGTAAGCTG GTGAGCCACT 3′,
5′ CAGTGTAATCTGGTGAGCCA 3′,
and
5′ CAGTGTAAGCTGGTG 3′.

27. (canceled)

28. A pharmaceutical composition comprising the oligonucleotide of claim 1 or salt thereof and a pharmaceutically acceptable solvent, diluent, carrier, salt or adjuvant.

29. The pharmaceutical composition of claim 28, wherein the pharmaceutical composition is formulated for oral or intravenous delivery.

30. (canceled)

31. A method of treating a subject having Fibrodysplasia ossificans progressiva (FOP), comprising: administering the oligonucleotide of claim 1.

32. The method of claim 31, wherein the patient is pre-screened for the 617G>A mutation

33. The method of claim 31, wherein the subject is a human.

34. A pharmaceutical composition comprising the oligonucleotide of claim 25 or salt thereof and a pharmaceutically acceptable solvent, diluent, carrier, salt or adjuvant.

35. The pharmaceutical composition of claim 34, wherein the pharmaceutical composition is formulated for oral or intravenous delivery.

36. A method of treating a subject having Fibrodysplasia ossificans progressiva (FOP), comprising: administering the oligonucleotide of claim 25.

37. A pharmaceutical composition comprising the oligonucleotide of claim 26 or salt thereof and a pharmaceutically acceptable solvent, diluent, carrier, salt or adjuvant.

38. The pharmaceutical composition of claim 37, wherein the pharmaceutical composition is formulated for oral or intravenous delivery.

39. A method of treating a subject having Fibrodysplasia ossificans progressiva (FOP), comprising: administering the oligonucleotide of claim 26.


SEQUENCE LISTING (31 sequences)

SEQ ID NO: 1 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
atctggtgagccactg

SEQ ID NO: 2 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
agctggtgagccactg

SEQ ID NO: 3 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
tgagccactgttcttt

SEQ ID NO: 4 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
taagccactgttcttt

SEQ ID NO: 5 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
gtgagccactgttctt

SEQ ID NO: 6 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
aacagtgtaatctggt

SEQ ID NO: 7 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
aacagtgtaatctgat

SEQ ID NO: 8 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
acagtgtaatctggtg

SEQ ID NO: 9 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
gtgtaatctggtgagc

SEQ ID NO: 10 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
gtgtaagctggtgagc

SEQ ID NO: 11 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
gtaatctggtgagcca

SEQ ID NO: 12 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
gtaagctggtgagcca

SEQ ID NO: 13 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
aatctggtgagccact

SEQ ID NO: 14 - DNA, 20 bp, synthetic construct
  source 1..20: mol_type = other DNA
  modified_base 1..4: mod_base = OTHER; note = RNA (2'-O-methoxyethyl-RNA)
  modified_base 17..20: mod_base = OTHER; note = RNA (2'-O-methoxyethyl-RNA)
  modified_base 1..20: mod_base = OTHER; note = Phosphorothioate bonds
  misc_feature 5..16: map = DNA
  misc_feature 17..20: map = RNA
gtgtaatctggtgagccact

SEQ ID NO: 15 - DNA, 20 bp, synthetic construct
  source 1..20: mol_type = other DNA
  modified_base 1..4: mod_base = OTHER; note = RNA (2'-O-methoxyethyl-RNA)
  modified_base 17..20: mod_base = OTHER; note = RNA (2'-O-methoxyethyl-RNA)
  modified_base 1..20: mod_base = OTHER; note = Phosphorothioate bonds
  misc_difference 5..16: note = DNA
gtgtaagctggtgagccact

SEQ ID NO: 16 - DNA, 20 bp, synthetic construct
  source 1..20: mol_type = other DNA
  modified_base 1..4: mod_base = OTHER; note = RNA (2'-O-methoxyethyl-RNA)
  modified_base 17..20: mod_base = OTHER; note = RNA (2'-O-methoxyethyl-RNA)
  modified_base 1..20: mod_base = OTHER; note = Phosphorothioate bonds
  misc_feature 5..16: note = DNA
cagtgtaatctggtgagcca

SEQ ID NO: 17 - DNA, 20 bp, synthetic construct
  source 1..20: mol_type = other DNA
  modified_base 1..4: mod_base = OTHER; note = RNA (2'-O-methoxyethyl-RNA)
  modified_base 17..20: mod_base = OTHER; note = RNA (2'-O-methoxyethyl-RNA)
  modified_base 1..20: mod_base = OTHER; note = Phosphorothioate bonds
  misc_feature 5..16: note = DNA
cagtgtaagctggtgagcca

SEQ ID NO: 18 - RNA, 18 nt, synthetic construct
  source 1..18: mol_type = other RNA
aacagtggctcaccagat

SEQ ID NO: 19 - DNA, 24 bp, synthetic construct
  source 1..24: mol_type = other DNA
tctggtcttccttttctggtacaa

SEQ ID NO: 20 - DNA, 19 bp, synthetic construct
  source 1..19: mol_type = other DNA
cccgacacactccaacagt

SEQ ID NO: 21 - DNA, 16 bp, synthetic construct
  source 1..16: mol_type = other DNA
agtggctcgccagatt

SEQ ID NO: 22 - DNA, 17 bp, synthetic construct
  source 1..17: mol_type = other DNA
cagtggctcaccagatt

SEQ ID NO: 23 - DNA, 15 bp, synthetic construct
  source 1..15: mol_type = other DNA
  modified_base 1..3: mod_base = OTHER; note = Locked Nucleic Acid
  modified_base 13..15: mod_base = OTHER; note = Locked Nucleic Acid
  modified_base 1..15: mod_base = OTHER; note = Phosphorothioate bonds
aacacgtctatacgc

SEQ ID NO: 24 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bonds
agcaatcatgataagc

SEQ ID NO: 25 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bond
gtcttgcggatggatt

SEQ ID NO: 26 - DNA, 15 bp, synthetic construct
  source 1..15: mol_type = other DNA
  modified_base 1..3: mod_base = OTHER; note = Locked Nucleic Acid
  modified_base 13..15: mod_base = OTHER; note = Locked Nucleic Acid
  modified_base 1..15: mod_base = OTHER; note = Phosphorothioate bond
ggttgcgccttttaa

SEQ ID NO: 27 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..16: mod_base = OTHER; note = Phosphorothioate bond
ttcatctagaacttcg

SEQ ID NO: 28 - DNA, 15 bp, synthetic construct
  source 1..15: mol_type = other DNA
  modified_base 1..3: mod_base = OTHER; note = Locked Nucleic Acid
  modified_base 13..15: mod_base = OTHER; note = Locked Nucleic Acid
  modified_base 1..15: mod_base = OTHER; note = Phosphorothioate bond
tactggagtgtcttg

SEQ ID NO: 29 - DNA, 20 bp, synthetic construct
  source 1..20: mol_type = other DNA
  modified_base 1..5: mod_base = OTHER; note = RNA (2'-O-methoxyethyl RNA)
  modified_base 16..20: mod_base = OTHER; note = RNA (2'-O-methoxyethyl RNA)
gtgtaagctggtgagccact

SEQ ID NO: 30 - DNA, 15 bp, synthetic construct
  source 1..15: mol_type = other DNA
  modified_base 1..5: mod_base = OTHER; note = RNA (2'-O-methoxyethyl RNA)
  modified_base 11..15: mod_base = OTHER; note = RNA (2'-O-methoxyethyl RNA)
cagtgtaagctggtg

SEQ ID NO: 31 - DNA, 21 bp, synthetic construct
  source 1..21: mol_type = other DNA
  modified_base 1..5: mod_base = OTHER; note = RNA (2'-O-methoxyethyl RNA)
  modified_base 17..21: mod_base = OTHER; note = RNA (2'-O-methoxyethyl RNA)
cagtgtaatctggtgaagcca